(19) United States
(12) Patent Application Publication
(10) Pub. No.: US 2008/0195096 A1
(43) Pub. Date: Aug. 14, 2008

(54) Orthopaedic Helical Coil Fastener and Apparatus and Method for Implantation Thereof

(75) Inventor: Hanspeter Frei, North Vancouver, CA (US)

Correspondence Address: Ralph A. Dowell of DOWELL & DOWELL P.C., 2111 Eisenhower Ave, Suite 406, Alexandria, VA, 22314, US

(73) Assignee: THE UNIVERSITY OF BRITISH COLUMBIA, Vancouver, BC (CA)

(21) Appl. No.: 11/665,367

(22) PCT Filed: Oct. 17, 2005
(86) PCT No.: PCT/CA05/01602

§ 371 Date: Jun. 13, 2007

Related U.S. Application Data
(60) Provisional application No. 60/618,561, filed on Oct. 15, 2004.

(51) Int. Cl. A61B 17/56; A61F 2/28

(52) U.S. Cl. 606/60; 623/16.11

(57) ABSTRACT

Provided herein is an implantable device and an expansion apparatus, method and system for implantation of the implantable device. In some embodiments, the device may include a helical coil having a contracted state, adapted for radial expansion and longitudinal contraction to an expanded state. The helical coil in the contracted state is adapted for positioning at a target site having walls defining an opening and is operable to engage the walls of the opening at said target site in the expanded state. The helical coil may also have an inner surface proximal to the longitudinal axis of the helical coil and an outer surface distal to the longitudinal axis of the helical coil, wherein the inner surface is operable to engage a fastener in the expanded state and wherein the outer surface defines a plurality of teeth extending radially outward to engage the walls of the target site.

### BACKGROUND OF THE INVENTION

Loosening and backing out of a fixation device can result in decreased structural integrity and increased maintenance and repair time. Furthermore, once a fastener as managed to work itself loose, wear and tear to the opening or space in the substrate within which it was received may prohibit securely refastening the fastener to the substrate. Similarly, bone fixation devices which cannot gain significant purchase in a bone substrate can result in clinical problems such as non-union or loss of corrections. Accordingly, much effort has been devoted to develop methods for preventing the failure of fixation devices. Accordingly, efforts are being made to determine the effects of screw design, depth of penetration and cement augmentation on pull out strength (J. G. Heller et al. J. Bone J. Surg [am] (1996) 78:1315-1321 and M. H. Cragg et al J. Spinal Disord. (1988) 1:287-294). Polymethylmethacrylate (PMMA) and more recently biodegradable calcium phosphate cements have been shown to increase the holding power of screws in bone (N. E. Motzkin et al J. Bone J. Surg [Br] (1994) 76:320-323 and B. C. Kleeman et al Clin. Orthop. (1992) 284:260-266). However, PMMA is exothermic upon polymerization and toxic monomers can cause bone necrosis, proliferation of fibrous tissue layers and other adverse biological responses (H. C. M. Amstutz et al Clin. Orthop. (1992) 276:7-18 and J. G. Heller et al J. Bone J. Surg. [Am] (1996) 78:1315-1321). Procedures, such as cement injection, can be more time intensive, as the cement generally requires 1-2 minutes of mixing and then a further wait of approximately 3 minutes (depending on the cement used and the temperature at which it is mixed) for the cement to increase in viscosity. Once cement is injected a further 2-3 minutes are often required before the screws can be inserted. In addition, it is often difficult to control cement flow, which can be further complicated by other factors, such as viscosity at time of injection, porosity of the bone, blood backflow, the pressure of the cement and the amount of cement lost at the injection site. As a result, if insufficient cement is injected its benefits may not be realized and if too much cement is injected it is possible fiat blood supply may be compromised or heat generated during polymerization can result in necrosis of the bone. Cement induced osteolysis or necrotic bone may impair the fixation and lead to eventual fastener loosening and failure. In the case of failure it is often difficult to remove cement from the bone and it is usually associated with excessive damage to the surrounding bone.

The use of bone cement and bone cement protocols in orthopaedic applications have been associated with bone cement implantation syndrome, which could ultimately result in death. When bone cement is packed into a bone, small pieces of bone or fat can enter the bloodstream. These particles may cause embolisms which could result in death of the patient. It is estimated that bone cement implantation syndrome occurs in 1 out of every 1000 bone cement procedures. Furthermore, the implantation of fixation devices in or between vertebrae (i.e. for spinal fixation procedures) may be particularly problematic. Such procedures must take into account the spinal cord and nerve roots, whereby if cement is in contact or exerting pressure on these structures during or after polymerizations damage or irritation of the spinal cord and nerve roots could cause neurologic symptoms. Often absorbable cements have similar disadvantages and usually do not provide sufficient strength.

The fixation of screws in osteoporotic bone can be particularly problematic. Fractures associated with osteoporosis are of significant concern and account for the majority of the expenditures for this condition. Stable internal fixation is required for most orthopaedic procedures, but such fixation in osteoporotic bone presents unique challenges. Screw loosening and subsequent implant failure are major complications. The ability of a screw to resist loosening is related to bone quality (O. R. Zindric et al Clinical Orthopaedics (1986) 203:99-112), while the holding power of a fixation device correlates with mineral density (T. C. Ryken et al Journal of Neurosurgery (1995) 83:325-329).

A number of cementless solutions have been proposed, such as interlocking screws (B. E. McKoy, 47^{th} Annual Meeting, Orthopaedic Research Society, Feb. 25-28, 2001, Session 19, Bone Mechanics II) and bone screw anchors (B. E. McKoy and Y. H. An Journal of Orthopaedic Research (2001) 19:545-547). Other bone implantation/fixation devices and methods are known in the art, for example, U.S. 2004/0181225, U.S. Pat. No. 5,084,050, U.S. Pat. No. 5,720,753, U.S. Pat. No. 6,656,184, U.S. Pat. No. 6,517,542 and U.S. Pat. No. 6,835,206. Helical anchors are generally well known, for example, U.S. Pat. No. 806,406, U.S. Pat. No. 3,983,736, U.S. Pat. No. 4,536,115, U.S. Pat. No. 5,312,214, U.S. Pat. No. 6,276,883, U.S. Pat. No. 6,494,657 and U.S. Pat. No. 6,860,691. Furthermore, helically wound springs have been described for use as tissue anchors (WO 01/08602) and helical coils have been described for use as surgical implants (U.S. 2004/0225361).

### SUMMARY OF THE INVENTION

In accordance with one aspect of the invention, there is provided an implantable device, wherein the device may include at least one helical coil having a contracted state, and following radial expansion an expanded state, wherein the helical coil is adapted for positioning at a target site within an opening in the contracted state and operable to engage the walls of the opening at said target site in the expanded state. The helical coil may also have an inner surface proximal to the longitudinal axis of the helical coil and an outer surface distal to the longitudinal axis of the helical coil, wherein the inner surface is operable to engage a fastener in the expanded state and wherein the outer surface defines a plurality of teeth. The device may include more than one helical coil. The device may also include a fastener. The helical coil or coils may be continuous. The fastener may be helical. The threads of the helical fastener may interdigitate with the threads on the inner surface of the helical coil.

In accordance with another aspect of the invention, there is provided an implantable orthopaedic device, the device including a helical coil having a contracted state, adapted for radial expansion and longitudinal contraction to an expanded state, wherein the helical coil when in the contracted state is adapted for positioning at a target site having walls defining an opening and is operable to radially expand to engage the walls of the opening at the target site in the expanded state; and the helical coil may have an inner surface proximal to the longitudinal axis of the helical coil and an outer surface distal to the longitudinal axis of the helical coil, wherein the inner surface is operable to engage a fastener in the expanded state and wherein the outer surface defines a plurality of teeth extending radially outward to engage the walls of the target site.

In accordance with another aspect of the invention, there is provided an orthopaedic device, including a helical coil in an expanded state, wherein the helical coil is radially expanded and is engaging the walls of the opening at a target site; and the helical coil is engaging a fastener and wherein the outer surface of the helical coil defines a plurality of teeth extending radially outward to engage the walls of the target site. The teeth may also have bone ingrowth to stabilize the helical coil and fastener in situ.

The device may include more than one helical coil. The device may also include a fastener. The helical coil or coils may be continuous. The fastener may be helical. The threads of the helical fastener may interdigitate with the threads on the inner surface of the helical coil. The implantable device may further include a compatible helical fastener, wherein compatibility is with respect to size thread pitch, composition i.e. compatible metals etc. The helical coil in the contracted condition in some embodiments may have an outer radial diameter of about less than or about equal to file outer diameter of the helical fastener. The helical coil in the contracted condition in some embodiments may have an outer radial diameter of about less than or about equal to the target site diameter. The device may be biocompatible. The device may be bioabsorbable or absorbable. The teeth of the helical coil may form an outer thread. The outer thread may form a sharp crest. The helical coil may be directional. The teeth may have a leading surface and a trailing surface. The leading surface may be sloped from the coil root to the crest. The plurality of teeth may be generally triangular shaped in a plane perpendicular to the longitudinal axis of the helical coil. Furthermore the radial spacing between teeth may vary. The plurality of teeth may also define a plurality of intervening notches and depending on the spacing of the teeth the size of the notches may vary. The plurality of intervening notches may facilitate radial expansion of the helical coil to the expanded state and the intervening notches may also facilitate bone ingrowth.

In some embodiments the device may have one or more coatings to promote bone ingrowth. The coating may be selected from one or more of the following: hydroxyapatite, bone morphogenic protein-2, retinoic acid and bisphosphates. The device may also have a porous surface to promote bone ingrowth.

The device may be made from a shape memory alloy. The shape memory alloy may be a nickel titanium alloy. The shape memory alloy may be characterized by an expanded state that occurs at or about body temperature. Alternatively, the device may be made from titanium, titanium alloys, 316L stainless steel, cobalt chrome alloys and non-absorbable and absorbable polymers.

In accordance with another aspect of the invention, there is provided an apparatus including: a tool body having a mandrel end for radially expanding a helical coil and defining a bore extending along its longitudinal axis; an insert coaxially disposed in said bore of said tool body and axially movable in said bore of said tool body between a first position and a second position; and a coil holder operably attached to the insert adjacent the mandrel end of the tool body, the coil holder including: a coil retainer operably attached to the insert and sized to fit the inner diameter of the helical coil in a contracted state; and a driver positioned distal to the insert and sized to pass through the inner diameter of the helical coil in a expanded state and sized to not to pass through the inner diameter of said helical coil in a contracted state. Furthermore, the axial movement of said insert from the first position to the second position may pull the coil holder toward the mandrel end of the tool body thereby forcing the helical coil onto the mandrel to radially expand the coil to the expanded state.

The axial force may be applied to the apparatus via a series of external threads on the insert operable to engage a series of internal threads on the tool body operable to move the insert axially from said first position to said second position or from said second position to said first position depending on the direction of rotation. The expansion apparatus may further include one or more handles for exerting rotational force on the insert relative to the tool body to produce axial movement of said insert relative to said tool body. The mandrel end may be operable to threadedly engage the helical coil.

In accordance with another aspect of the invention, there is provided an expansion apparatus, the apparatus including: a tool body defining a bore extending along its longitudinal axis; an insert coaxially disposed in said bore of said tool body and axially movable in said bore of said tool body between a first position and a second position; and a coil holder operably attached to the insert so that the coil holder extends beyond bore of the tool body, the coil holder including: (i) a coil retainer operably attached to the insert and sized to fit the inner diameter of the helical coil in a contracted state; and a mandrel positioned distal to the insert and sized to pass through the inner diameter of the helical coil in a expanded state and sized to not to pass through the inner diameter of said helical coil in a contracted state. Furthermore, the axial movement of said insert from the first position to the second position may pull the coil holder toward the tool body thereby forcing the helical coil onto the mandrel to radially expand the coil to the expanded state.

An axial force may be applied to the apparatus via a series of external threads on the insert operable to engage a series of internal threads on the tool body operable to move the insert axially from said first position to said second position or from said second position to said first position depending on the direction of rotation. The expansion apparatus may hither include one or more handles for exerting rotational force on the insert relative to the tool body resulting in axial movement of said insert relative to said tool body. Alternatively, axial movement may be achieved with gearing, hydraulically or via additional means for achieving such movement. The mandrel may be operable to threadedly engage the helical coil.

In accordance with another aspect of the invention, there is provided an expansion apparatus as described herein may further include a stabilizer operably mounted on the tool body to control rotational movement of the helical coil during expansion.

In accordance with another aspect of the invention, there is provided a surgical method for helical coil expansion in situ, the method including: positioning of a helical coil in a contracted state and an expansion tool operably engaging the helical coil at a target site; radially expanding the helical coil to an expanded state with the expansion tool; removing the expansion tool; and threadedly engaging the helical coil with a threaded fastener.

In accordance with another aspect of the invention, there is provided a method including: positioning of a helical coil in a contracted state and an expansion tool operably engaging the helical coil at a target site; radially expanding the helical coil to an expanded state with the expansion tool; removing the expansion tool; positioning of a second helical coil in a contracted state and an expansion tool operably engaging the helical coil at a second target site; removing the expansion tool; and threadedly engaging the helical coil with a threaded fastener.

The method may further include the positioning of additional coils at additional target sites. The second target site or additional target site may be within a previously positioned and expanded coil. The second target site or additional target site may be stacked adjacent a previously positioned and expanded coil.

In accordance with another aspect of the invention, there is provided a system for delivering a helical coil in a predrilled opening in a bone to provide additional resistance to pull out for a helical fastener, the system including: at least one helical coil in a contracted state; an expansion tool operably engaging the helical coil to position the helical coil at a target site within the bone and to subsequently radially expand the helical coil to am expanded state allowing for removal of the expansion tool; and a threaded fastener threadedly inserted into the helical coil in the expanded state.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1A is a perspective view of a first embodiment of a helical coil in a contracted state.

FIG. 1B is a perspective view of the helical coil in an expanded state.

FIG. 2A is a top view of the helical coil of 1A.

FIG. 2B is a side view of the helical coil in 1A and 2A.

FIG. 2C is a side cross-sectional view of the coils of 1A, 2A and 2B.

FIG. 3A is an end view of the helical coil according to an alternative embodiment in a contracted state.

FIG. 3B is a cross-sectional side view of the helical coil shown in FIG. 3A taken along cross-section line B-B.

FIG. 3C is a side view of the helical coil shown in FIG. 3A.

FIG. 3D is a perspective view of the coil shown in FIG. 3A.

FIG. 4A shows an end view of a helical coil with alternative designs for teeth and notches.

FIG. 4B shows a perspective view of the helical coil shown in 4A.

FIG. 4C shows a side view of the helical coil shown in 4A.

FIG. 5 shows alternative helical coil designs with two coil projections (A), four helical coil projections (B) and three helical coil projections (C).

FIG. 6A shows a cross-section of a helical fastener according to the first embodiment shows in FIGS. 1 and 2.

FIG. 6B shows a helical fastener engaging the helical coil of 6A in an expanded state.

FIG. 7A shows a cross-sectional side view of an expansion apparatus engaging a helical coil in a contacted state.

FIG. 7B shows a cross-sectional side view of the helical coil in 7A in an expanded state.

FIG. 7C shows a cross-sectional side view of a helical fastener engaging the helical coil of FIG. 7B.

FIG. 8A shows a cross-sectional side view of an expansion apparatus engaging a helical coil in a contracted state, wherein the stabilizer apparatus has been deployed to engage the helical coil.

FIG. 8B shows a cross-sectional view of the expansion apparatus shown in FIG. 5A with the stabilizer apparatus not deployed and not engaging the helical coil.

FIG. 9A shows a cross-sectional side view of an expansion apparatus engaging a helical coil in a contracted state.

FIG. 9B shows a cross-sectional view of the expansion apparatus shown in FIG. 8A.

FIG. 10 shows a graph comparing the pull-out force required in a vertebrae for a pedicle screw alone as compared to a helical coil.

### DETAILED DESCRIPTION OF THE INVENTION

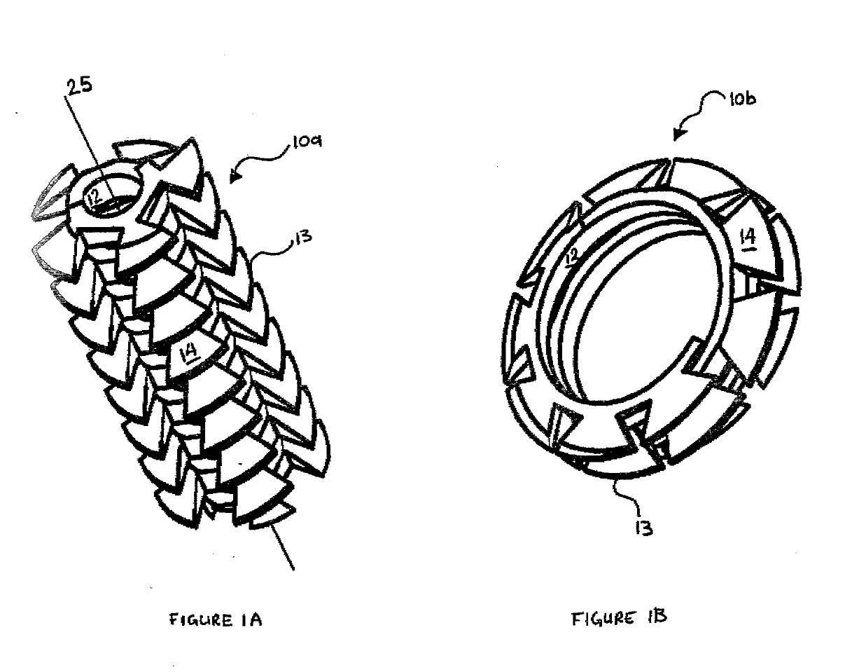

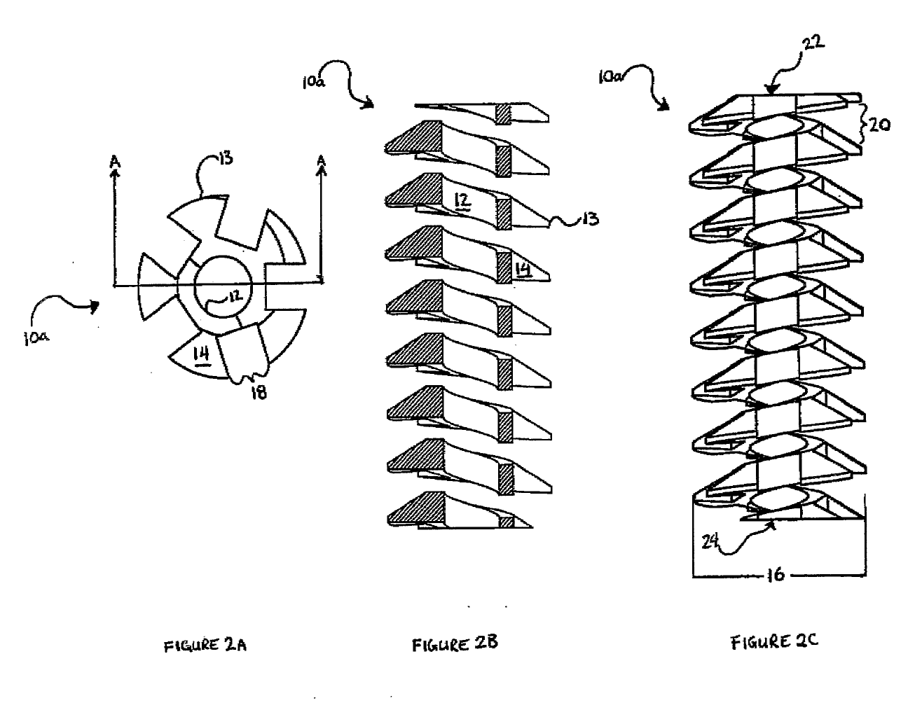

FIG. 1A shows a first embodiment of a helical coil in a contracted state. The coil is generally shown at 10a wound around a longitudinal axis 25. An inner surface 12 of the helical coil is shown proximal to the longitudinal axis of the helical coil. Whereas, the outer surface 13 defines a plurality of teeth or protrusions 14. Still referring to FIG. 1A, the outer surface of the helical coil 13 defines a series of notches or openings 18 between adjacent teeth 14. FIG. 2B shows the helical coil of the first embodiment shown in FIG. 1A in an expanded state. The coil is shown generally at 10b wound around the longitudinal axis 25. The inner surface of the coil 12 and teeth 14 are also shown. It is apparent from a comparison of FIGS. 1A and 1B that the expansion of the helical coil results in a reduction of the notched opening between adjacent teeth 14 potentially allowing for the teeth to grab adjacent substrate and thereby improve fixation of the implantable device.

FIG. 2A shows a top view of a first embodiment of the helical coil shown generally at 10a in a contracted state as in FIG. 1A. The inner surface 12 as defined by the helical coil is shown proximal to the axis of the coil. Similarly the outer surface 13 (distal to the axis of the coil) defines a plurality of teeth 14 spaced by notches 18. Also shown in FIG. 2A is an outside diameter 16 and cross-sectional line AA. FIG. 2C also shows the contracted helical coil in a side view with coil tarn space 20 and outside diameter 16 also shown. In this embodiment the teeth 14 have a generally triangular shape in the end view (FIG. 2A) as well as in the side view (FIG. 2B) which may be produced if a level or chamfer is cut into generally rectangular cross-sectioned coil. Also shown in FIG. 2C is a leading end 22 and trailing end 24.

FIG. 2B shows a side cross-sectional view taken along line AA in FIG. 2A of the contracted helical coil of the first embodiment. Teeth are shown at 14, inner surface 12 and the coil turn space 20 is also shown.

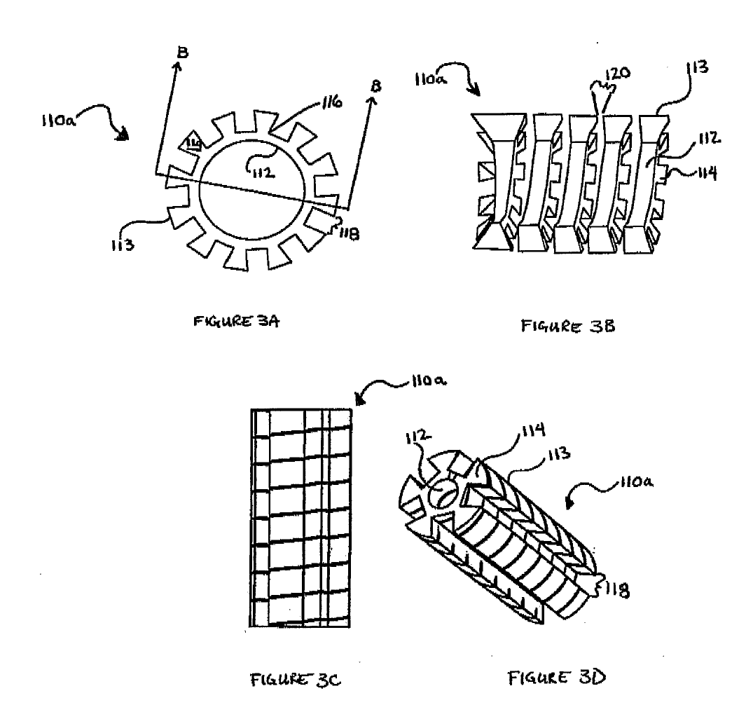

FIG. 3A shows a second embodiment of the helical coil in an end view generally at 100a (in a contracted state). Teeth 114 cross-sectional line B-B. In FIG. 3B, the helical coil of the second embodiment shown in FIG. 3A is shown in cross-section taken along line B-B with inner surface 112 and teeth 114 which are generally rectangular in cross-section as compared to the embodiment shown in FIGS. 1 and 2. The inner surface 112 is very similar to the first embodiment shown in FIGS. 1 and 2. However, because of the differences in the cross-sections of the teeth between the first and second embodiments the coil turn space 120 is considerably reduced in the cross-section. In FIG. 3C, the helical coil is shown in a side view and in FIG. 3D the alternative embodiment helical coil is shown in perspective view also in the contracted state.

Although the inner surface 112 is very similar in this second embodiment as the one in the first embodiment the outer surface 113 is significantly different, whereby the teeth 114 have a much wider cross-section at the distal edge of the coil in comparison to the first embodiment. Furthermore, in FIG. 3A it will be noted that the top view of the teeth 114 have a tooth base 116 which is wider than that of the first embodiment. Furthermore, the notches 118 are shown in FIG. 3A.

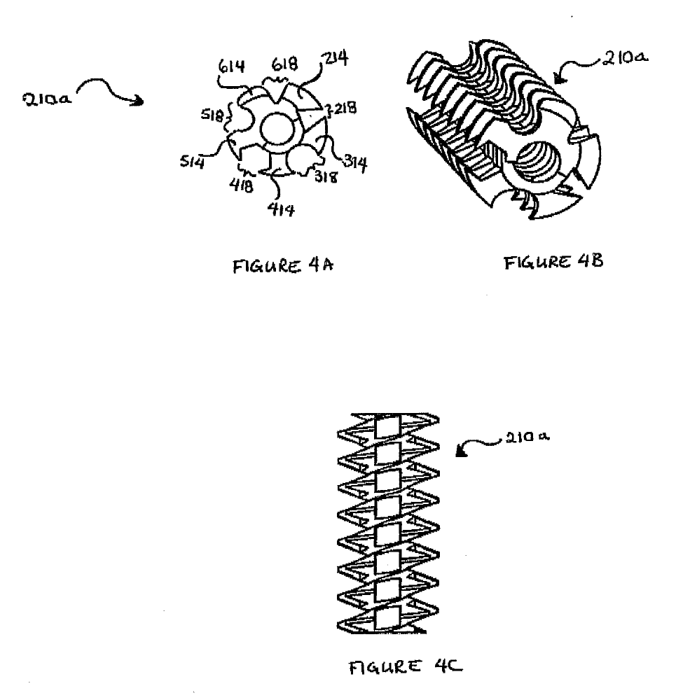

FIG. 4 shows a variety of additional embodiments generally at 210a with variation in the shape of the teeth and intervening notches. FIG. 4A is an end view showing various helical anchor arrangements wherein teeth 214, 314, 414, 514 and 614 and notches 218, 318, 418, 518 and 618 of various possible designs are combined in one coil. FIG. 4B shows the helical coil of FIG. 4A in a perspective view and FIG. 4C shows a side view of the coil in FIGS. 4A and 4B. The alternative designs for teeth and notches shown in FIGS. 4A-C and in embodiments one and two may be taken in any combination thereof depending on the particular use. Furthermore, the alternative designs for the various teeth and notch arrangements may be chosen one at a time in designing a helical coil, again depending on the particular use. In general, the alternatives shown in FIG. 4 are primarily illustrative of alternative designs for the teeth and notches of a helical coil.

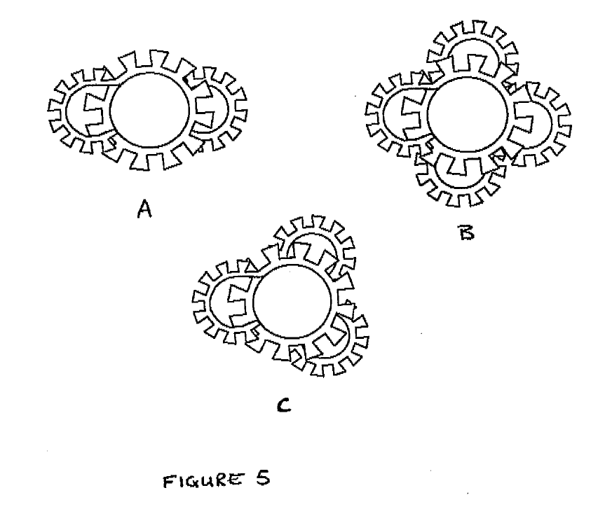

FIG. 5 shows end views of a helical coil similar to the second embodiment shown in FIGS. 3A-D where the coil pattern has been disrupted to create 2, 4, and 3 offshoots respectively (A-C). These offshoots may be positioned proximal or distal to the cortical bone and generally act to expand the effective external diameter of the helical coil while still providing a threaded surface for engagement with a helical fastener.

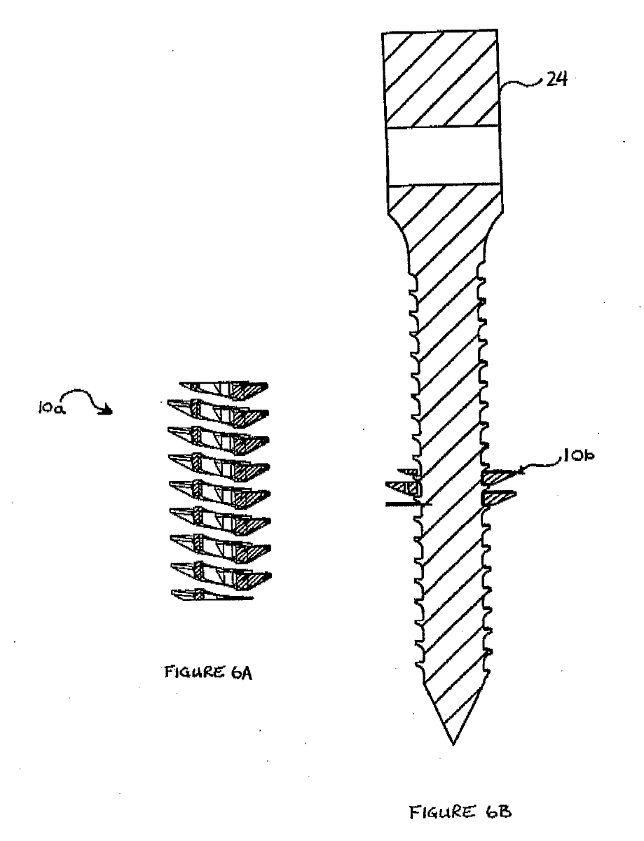

FIG. 6A cross-sectional side view of a helical coil 10a according to a first embodiment and in a contracted state. FIG. 6B shows the helical coil 10b of FIG. 6A also in a cross-sectional side view, but in an expanded state and threadedly engaging a helical fastener 24.

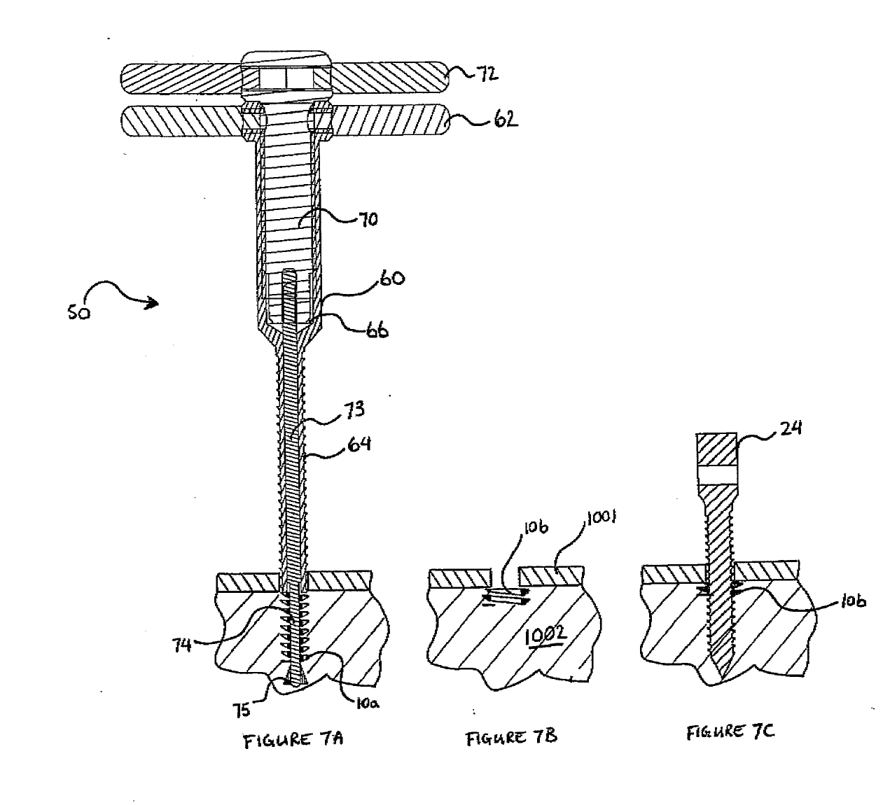

FIG. 7A shows a first embodiment of an expansion apparatus generally at 50, wherein the apparatus includes a tool body 60 having a mandrel end 64 operable to readily expand a helical coil and the tool body defining a bore 66 extending along the longitudinal axis of the tool body. Also shown in FIG. 7A is the tool body handle 62, insert 70 which is coaxially disposed in the bore of the tool body 60 and insert handle 72. Attached to the insert 70 via the bore 66 is the coil holder 73 which is operable attached to the insert adjacent the mandrel end 64 (shown here threaded) of the tool body 60. The coil holder 73 is comprised of a coil retainer 74 operably attached to the insert and to a driver 75 shown at the distal end of the apparatus. Furthermore, a helical coil 10a in a contracted state is shown positioned on the coil retainer 74 adjacent the driver 75. FIG. 73 shows the expanded helical coil in cross-section positioned just below the cortical bone 1001 within the trabecular bone 1002, whereby the expanded outer diameter is greater than the cortical bone opening. FIG. 7C shows the helical coil of 7B threadedly engaging a helical fastener 24 also shown in cross-section.

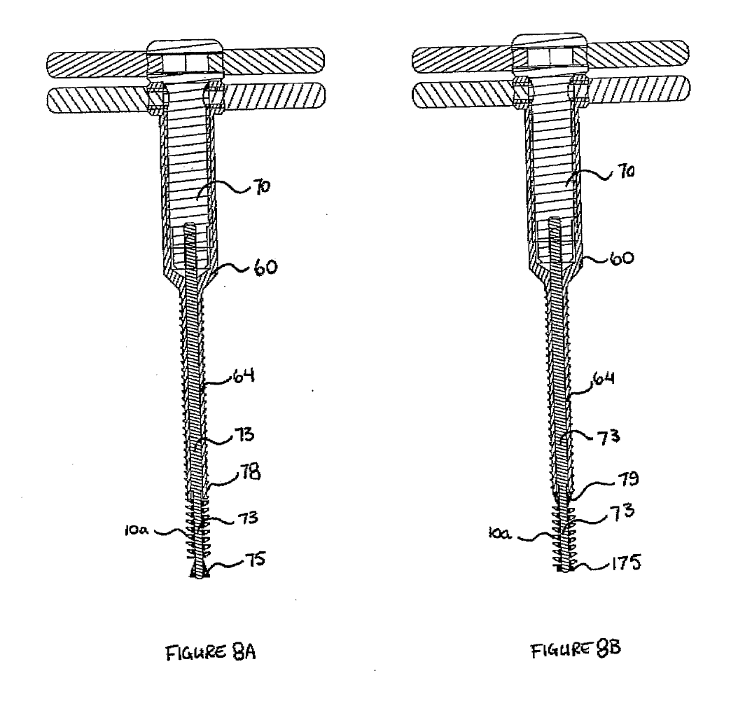

FIG. 8A shows the expansion apparatus of FIG. 7A showing a driver 75 with a tapered cross-section so that the helical coil 10a may be forced over the driver and into an expanded state. Furthermore, the helical coil 10a may threadedly engage the threads of the tool body 78. FIG. 8B shows an alternative embodiment whereby the driver 175 has a surface perpendicular to the coil holder 73 which forces the helical coil 10a onto the tapered end 79 of the tool body for threaded engagement with the threads of the tool body. Both FIGS. 8A and 8B show the expansion apparatus in a first position whereby the helical coil 10a is an a contracted state. Rotational movement of the insert 70 relative to the tool body 60 results in movement of the apparatus to a second position whereby the helical coil is forced onto the mandrel end 64 to expand the helical coil (not shown).

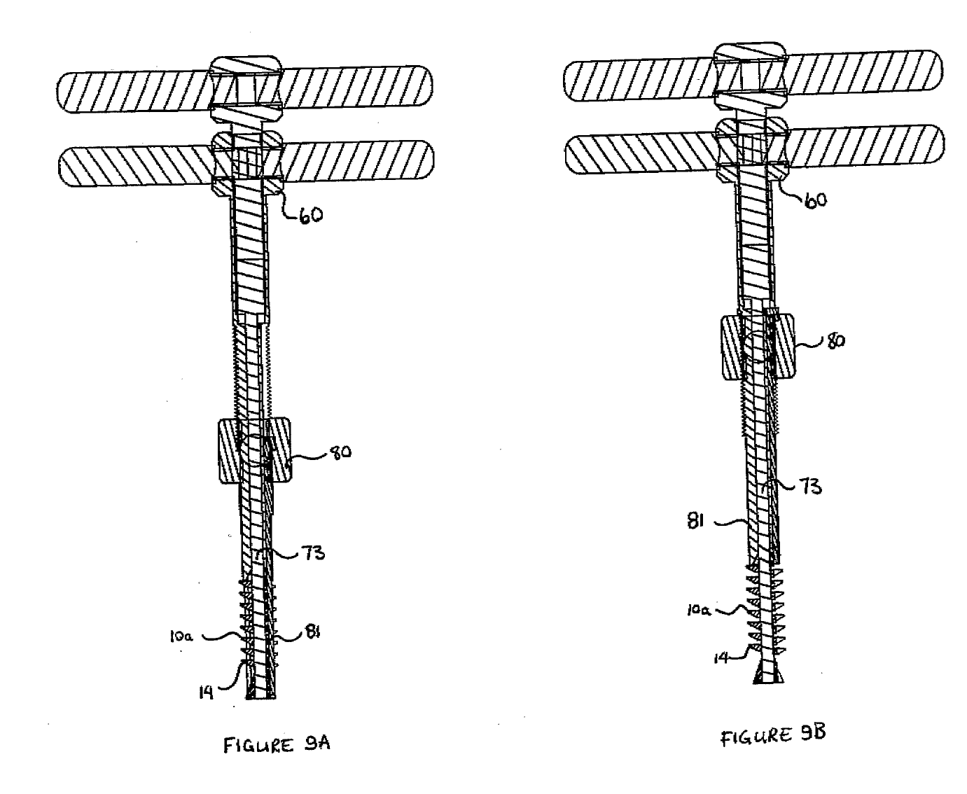

FIG. 9A shows an alternative file expansion apparatus with an additional stabilizer 80 in a deployed position engaging the helical coil 10a so that the stabilizer arms 81 engage the helical coil 10a between its teeth 14 to control rotational movement of the helical coil relative to the holder. FIG. 9B shows the stabilizer 80 in a second position whereby the stabilizer arms are not engaging the helical coil 10a. In the present embodiment the stabilizer threadedly engages the outer surface of the tool body 60.

### Materials

The helical coils and helical fasteners described herein may be selected from appropriate materials depending on the particular application (i.e. strength, weight, durability, compatibility, etc.). Biocompatible materials as described herein may be selected but not limited to the following: titanium, titanium alloys, 316L stainless steel, cobalt chrome alloys and non-absorbable and absorbable polymers as known in the art. In deciding what materials may be chosen for an implantation device as described herein a further consideration is the compatibility of the materials used in the helical coil with the materials used in the helical fasteners and also with the materials used in other devices which may be used in conjunction with the implant devices described herein. Materials are potentially considered compatible if they do not create galvanic corrosion which results when dissimilar metals are used. Persons of skill in the art would be able to match materials accordingly.

Furthermore, the compatibility of a device as described herein may also include compatibility of threads on a helical fastener (i.e. pitch size, depth, vertical spacing, etc.) to match the helical coil which it is intended to match up with. IN addition, the internal threads on an expanded first helical coil may be compatible to engage the external threads on a second coil inserted and expanded within the first helical coil etc.

Furthermore, the implantable devices described herein may be coated with a porous and bioactive material or a combination thereof to allow bone growth onto the device and to promote bone growth into any notches or other openings or spaces surrounding the device (collectively bone in-growth). For example, one or more of hydroxyapatite, bone morphogenic protein-2 (BMP-2), retinoic acid and biophosphonates may enhance bone in-growth. Alternatively, the surface of the device (helical coil or helical fastener) could be porous to similarly encourage bone growth and promote fixation of the device within the bone. Alternatively, a combination of coatings and surface preparation s 9 for example, etched or porous surfaces) may be used on the entire surface of the device or restricted to particular regions of the device.

Alternatively, the device may be made of a bioabsorbable polymer which as it becomes absorbed may be replaced by new bone growth over time. Where the implantable device is made from a bioabsorbable or resorbable material, the rate at which the material breaks down will depend on the particular application of the implantable device. Compositions for bioabsorbable materials may be customized to the particular physical parameters required by the implantable device in review of the particular intended use. Bioabsorbable materials may, for example, be chosen from one or more of the following: homopolymers or copolymers of lactide; glycolactide; polydioxone; trimethylene; carbonate; polyorthoesters; polyethylene oxidel; or other polymer materials or blends thereof.

In alternative embodiments, the device may also be formed out of shape memory alloys (SMA) such as nickel titanius (NiTi) shape memory alloys (Nitinol), whereby the alloy device could be programmed to be in the contracted state at one temperature (i.e. either below or above body temperature) and in the expanded state at or around body temperature. Thus, potentially allowing for self-expansion of a helical coil at a desired target site by merely allowing the coil to come to body temperature. Depending on the exact composition and whether or not the expanded coil is in the martensite phase or the austenite phase nickel titanium SMAs can lave a variety of very useful properties such as dampening and vibration attenuation, which could aid in dampening peak stresses between tire bone and an articular prosthesis. Furthermore, the low elastic modulis, high fatigue, ductile and high resistance to wear of NiTi alloys may also prove useful in various orthopaedic applications. Furthermore, NiTi alloys are non-magnetic, thereby permitting MRI imaging. Solid NiTi alloys may be manufactured by a double vacuum melting process (after formulation of raw materials alloy is vacuum induction melted followed by vacuum arc remelting) and ingots can be hot worked and cold worked into a wide range of sizes and shapes. Furthermore, porous NiTi can be made by sintering or using self-propagating high temperate synthesis (ignition synthesis) which may be useful in promoting bone in-growth. In order to program a NiTi SMA the material is molded into the desired shape and a heat treatment is then applied to set the specimen into its final shape. The heat treatment parameters (temperature and time, etc.) will depend on the desired characteristics of the final product, and is followed by rapid cooling (such as water quenching or rapid air-cooling).

Many NiTi SMAs have a soft martensite phase when dropped below a transition temperature and a hard austenite phase when raised above the transition temperature. By cooling a SMA implant device to the martensite phase, it may be advantageous to have a helical coil in a contracted state whereby it can be positioned at a target site and when heated to body temperature to the austenite phase the helical coil would harden and through its shape memory revert to the expanded state. The resultant change in geometry in situ without the requirement for additional expansion procedures may be advantageous. Alternatively, the coil may be cooled to take on the expanded state.

Where it may not be possible or advantageous to store a SMA helical coil at or near a temperature which maintains the helical coil in a contracted state, two way shape memory training may be applied. Whereby the helical coil could be cooled or heated just prior to the orthopaedic procedure (depending on the alloy's properties and training) so that the coil takes on a contracted state when actually needed. Once inserted a helical coil could be taken to body temperature by active or passive means to produce a helical coil in its expanded state at a desired target site.

In other applications shape memory alloys used for implantable devices, as described herein, may be matched to the operating temperatures of the substrate in which they are implanted whereby a configuration change may be matched to changes in configuration of the surrounding substrate due to a temperature change.

A potentially wider ranges of materials may be available for more orthopaedic uses described herein.

### Uses

Depending on the design of the implantable devices described herein and the specific properties associated herewith (materials chosen, specific geometry, size and other properties selected in the design of the device), many applications are possible. For example, fracture fixation in normal and osteoporotic bone, the fixation of pedicle screws in vertebrae for the secure fixation of posterior fixation devices used in vertebral fusion applications, the use as bone anchors for the fixation of tendons and ligaments to bone, the use with bone screws to secure plates and rods in general trauma procedures, the use as interbody cages, the use in dental and orthodontic applications including implants and dental crowns, and in expandable stents for use in vascular and other medical procedures, etc.

Furthermore, use of the implantable device described herein may be achieved via a relatively small incision hole size to fit a helical coil as described herein for implantation into a target substrate. Furthermore, the implantation devices described herein may be applied with arthroscopic procedures via a narrow cannula depending on the particular application and the design of the implantable device. Because of the contracted and expanded states of the implantation device the opening required to position the contracted helical coil at a target site may be reduced. Upon expansion to an expanded state, the helical coil may then exert force on the target substrate adjacent the device to resist migration and pull out forces.

Potential substrates for example may include bone and cartilage for orthopaedic uses veins and arteries for vascular uses, ureters, etc. in urologic uses, and metal, plastic, wood, concretes drywall, etc. for structural and ornamental uses. The present devices may be of particular benefit in non-uniform substrates like bone, where the device may be able to expand at a target site and subsequently form stable interactions with the bone substrate.

The expansion of the helical anchors described herein may be performed in various ways to further expand the outer diameter of the helical coil. For example, as shown in FIG. 5A-C, the helical coil may be expanded to provide offshoots which further expand the outer diameter of a single helical coil and may potentially increase the pull out resistance of the implantable device. Initial implantation and expansion may be achieved as described herein. However, additional tools and helix designs may be useful to achieve the further radial expansion of offshoots from the central helix. To promote offshoot formation the helical coil may be engineered to have discontinuities or weaknesses at specific positions within the helical coil so that a fold or bend may result in the helical coil to produce an offshoot.

It an alternative embodiment, a first helical coil may be expanded at a target site and rather than receiving a helical fastener a further helical coil in a contracted state may be positioned within the previously expanded helical coil and subsequently expanded to further expand the diameter of the first helical coil. Such an expansion may be repeated multiple times prior to insertion of a helical anchor. Such a strategy would further expand the effective external diameter of the implantable device thereby potentially improving resistance to pull out forces and potentially providing greater stability to the device within the substrate. Furthermore, helical coils may be stacked end-to-end or spaced apart depending on the length of the helical fastener used and the depth of the opening in the substrate. Stacking may allow for greater helical fastener to helical coil interactions and potentially provide for a more stable insert and potentially greater opportunities for bone in-growth into the helical coil.

### Expansion

Expansion of the helical coil may be achieved via various methods and means depending on the design of the coil and the potential application. As described above, shape memory alloy helical coils may expand in response to a temperature change and require no intervention with an expansion tool other than potentially a heating or cooling device. In alternative embodiments a helical coil may be loaded under torsion and held in a contracted state by an insertion tool and not expanded until released by the insertion tool at a target site. Such an embodiment would require some degree or resilience in the helical coil.

In an alternative embodiment a non-resilient coil may be deformed with the use of an expansion tool, which may take various forms. For example, an expansion tool may push or pull the helical coil onto a mandrel which itself way alternatively be threaded resulting in an expanded helical coil having threads compatible with a helical fastener. Further embodiments are described herein in greater detail. Alternatively, expansion of a helical coil may occur by the insertion of a helical fastener into either a partially expanded or contracted helical coil. In other words, initial expansion may have occurred following positioning of the helical coil at a target site and further expansion may result from the insertion of a helical fastener or the coil may be expanded by the fastener alone. As described above, a first expanded helical coil may also be further expanded by the insertion and expansion or one or more additional helical coils within the inner diameter of the first expanded helical coil prior to insertion of a helical fastener. Such multi coil expansion may be aided by compatible threads on the external and internal surface of the helical coils.

Alternatively, a helical coil may be expanded by either pulling or pushing a punch axially through the inner diameter of the unexpanded helical coil to produce an expanded helical coil.

Expansion in the coil being changed from a contracted state having an insertion diameter to an expanded state having a retention diameter. Furthermore, helical expansion may result in both radial expansion of the coil, a pinching together of the teeth, and associated rotational movement of the coil, which may all further aid in forming a solid interaction with the surrounding substrate.

### Design Variations

Circumferential protrusions or teeth on the helical coil may be spaced by variously sized notches at the outer radial circumference of the helical coil, which may provide secondary fixation of the coil into the surrounding substrate. As a helical coil is expanded the teeth may be forced together thereby aiding in the expansion of file helical coil and potentially biting into the substrate into which the helical coils are expanded. Furthermore, the openings or notches between the teeth may facilitate bone in-growth to further enhance the stability and pull out strength of the implantable device. In an orthopaedic application, the teeth may extend into the trabecular bone to enhance fixation, which in some embodiments may be aided by a pointed or sharpened outer radial surfaces to aid passage through surrounding bone during rotational movement of the helical coil in the radial expansion phase.

In some embodiments the teeth may be articulated so that they are capable of independent movement relative to the coil. Furthermore, the number of notches and teeth, their relative spacing, shape, etc. may vary. Additionally, the helical coil may have a taper or irregular diameter along its length. In further embodiments the outer edge or teeth of the helical coil may be beveled or chamfered or alternatively shaped to produce a shaped thread. For example, the external edge of the teeth may form a sharp thread as shown in FIGS. 1A and 1B.

In contrast to the use of bone cements, the embodiments described herein may be inserted in less time and with fewer steps than procedures using cements. This may be in part due to the reduced preparation time and potential delays in awaiting cement fixation or polymerization. The helical coils described herein may also stimulate bone growth at insertion sites rather an bone necrosis which is possible with many bone cements.

### Manufacture of a Helical Coil

The helical coils described herein may be produced via various methods depending on the particular design and materials chosen.

For example in one possible method, a rectangular (or other shaped) rod may be wound around a cylinder or round rod having an outer diameter equal to the desired inner diameter of the resulting helical coil in a contracted state. The outer diameter of the helical coil may be calculated by taking 2 times the rectangular (or other shaped) rod's width plus the inner diameter of the resulting helical coil. The height of the rectangular (or other shaped) rod may be used to determine the pitch of the resulting helical coil. A chamfer or bevel may be machined long the length of the rectangular rod to account for the expansion of the material during bending (winding around the cylinder or round rod). After winding of the rectangular (or other shaped) rod, notches or cuts of various shapes may be machined (cut) along the length of the helical coil on the outer periphery (distal to the axis of the coil) to produce a plurality of teeth separated by notches, and both ends of the helical coil may be squared off or flattened.

Alternatively, a rod having an outer diameter approximately equivalent to the resulting outer diameter of a desired helical, coil may be cut to produce an outer thread having a pitch and dimensions to match the helical fastener with which it is to be paired. The outer thread of the helical coil may be cut somewhat deeper than that of the helical fastener and a thin flat parting tool may be used to make a further deep helical cut in the outer thread so that the cut is slightly deeper than the desired inner diameter of the helical coil. The threaded rod may then be drilled to produce a longitudinal bore in the rod with the desired inner diameter. As a result of the deep helical out the rod with the thread produces a helical coil when the inner bore is drilled. Finally, notches or cuts of various shapes may be machined (cut) along the length of the helical coil on the outer periphery (distal to the axis of the coil) to produce a plurality of teeth separated by notches on the flights of the helical coil.

Alternatively, helical coils in accordance with embodiments described herein, may be made by one or more other methods know in the arts. For example coils may be produced by die-casting or forged or via a combination of methods depending on the desired end product.

### EXAMPLE 1

### Comparison of Pull-Out Force Required for a Bone Screw in a Vertebrae with and without the Expandable Helical Coil

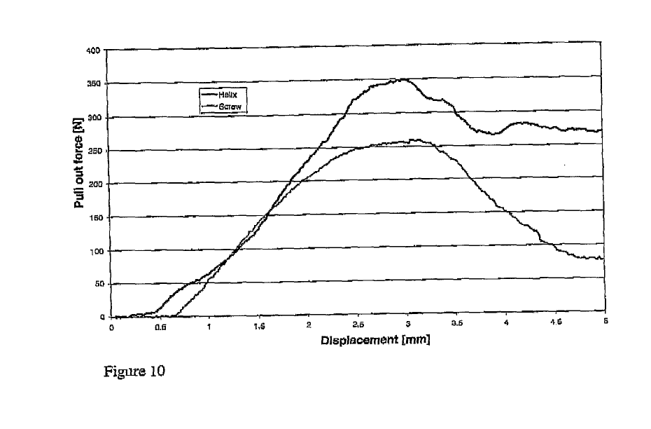

The fixation strength of an expandable helix in trabecular bone with a thin cortex was determined with a pull out test (see FIG. 10). A 6.3 mm hole was drilled into a vertebral body through which a helix was inserted. After expansion of the helix (6 mm to 9 mm) a pedicle screw with a 6 mm diameter and length of 45 mm (Synthes Spine, Paoli, Pa.) was inserted. As a result of the drill hole size the pedicle screw did not gain purchase within the vertebral body. The vertebral body was then inserted in a metal tube to prevent displacement of the vertebra in the direction of the pull out force. The screw head was then attached to an actuator on the test machine through a hole in the metal tube retaining the vertebra. A pull out displacement was applied until failure of the helix and pull out force was recorded. The pull out strength of the helix was compared to the pull out strength of the same screw without the helix, whereby the screw was placed in a 2 mm hole produced by a blunt pedicle probe drilled into the same vertebra at a different position prior to insertion of the screw. The pull out strength was determined as described above.

Pull out testing was performed using a modified foam testing method, ASTM standard F543-02 Annex A3 “Test Method for Determining the Axial Pullout Strength of Medical Bone Screws”.

As seen in FIG. 15, the maximum pull out strength of the screw fixed with the helix was 348 N (newtons) in contrast to the maximum pull out strength of the screw alone which was 254 N. Thus a 37% greater pull out force was required in the screw helix complex (where the screw did not contribute to pull out resistance) relative to a screw alone. Accordingly, it would be expected that further pull out resistance would be expected where the predrilled hole allowed for interaction of the screw with the substrate.

While specific embodiments in accordance with the invention have been described and illustrate such embodiments should be considered illustrative of the invention only and not as limiting the invention as construed in accordance with the accompanying claims.

1. An implantable orthopaedic device, the device comprising:
(a) a helical coil having a contracted state, adapted for radial expansion and longitudinal contraction to an expanded state, wherein the helical coil when in the contracted state is adapted for positioning at a target site having walls defining an opening and is operable to radially expand to engage the walls of the opening at the target site in the expanded state; and
(b) the helical coil having an inner surface proximal to the longitudinal axis of the helical coil and an outer surface distal to the longitudinal axis of the helical coil, wherein the inner surface is operable to engage a fastener in the expanded state and wherein the outer surface defines a plurality of teeth extending radially outward to engage the walls of the target site.

2. The implantable device of claim 1, wherein the inner surface is operable to engage a helical fastener in the expanded state.

3. The implantable device of claim 2, further comprising a compatible helical fastener.

4. The implantable device of claim 3, wherein the helical coil in the contracted condition has an outer radial diameter of about less than or about equal to the outer diameter of the helical fastener.

5. The implantable device of claim 1, wherein the device is biocompatible.

6. The implantable device of claim 5, wherein the device is bio absorbable.

7. The helical coil of claim 1, wherein the teeth of the helical coil form an outer thread.

8. The helical coil of claim 7, wherein the outer thread forms a sharp crest.

9. The helical coil of claim 1, wherein the helical coil is directional.

10. The helical coil of claim 9, wherein the teeth have a leading surface and a trailing surface.

11. The helical coil of claim 10, wherein the leading surface is sloped from the coil root to the crest.

12. The implantable device of claim 1, wherein the plurality of teeth are generally triangular shaped in a plane perpendicular to the longitudinal axis of the helical coil.

13. The implantable device of claim 1, wherein the plurality of teeth define a plurality of intervening notches.

14. The implantable device of claim 12, wherein the plurality of intervening notches facilitate radial expansion of the helical coil to the expanded state.

15. The implantable device of claim 13, wherein the intervening notches facilitate bone ingrowth.

16. The implantable device of claim 1, wherein the device has a coating to promote bone ingrowth.

17. The implantable device of claim 16, wherein the coating is selected from one or more of the following: hydroxyapatite, bone morphogenic protein-2, retinoic acid and bisphosphates.

18. The implantable device of claim 1, wherein the device has a porous surface to promote bone ingrowth.

19. The implantable device of claim 1, wherein the device is made from a shape memory alloy.

20. The implantable device of claim 19, wherein the shape memory alloy is a nickel titanium alloy.

21. The implantable device of claim 19, wherein the expanded state occurs about body temperature.

22-30. (canceled)

31. A surgical method for helical coil expansion in situ, the method comprising:
(a) positioning of a helical coil in a contracted state and an expansion tool operably engaging the helical coil at a target site;
(b) radially expanding the helical coil to an expanded state with the expansion tool
(c) removing the expansion tool; and
(d) threadedly engaging the helical coil with a threaded fastener.

32. A surgical method for helical coil expansion in situ, the method comprising:
(a) positioning of a helical coil in a contracted state and an expansion tool operably engaging the helical coil at a target site;
(b) radially expanding the helical coil to an expanded state with the expansion tool;
(c) removing the expansion tool;
(d) positioning of a second helical coil in a contracted state and an expansion tool operably engaging the helical coil at a second target site;
(e) removing the expansion tool; and
(f) threadedly engaging the helical coil with a threaded fastener.

33. The method of claim 32, further comprising the positioning of additional coils at additional target sites.

34. The method of claim 32, wherein the second target site or additional target site is within a previously positioned and expanded coil.

35. The method of claim 32, wherein the second target site or additional target site is stacked adjacent a previously positioned and expanded coil.

36. A system for delivering a helical coil in a predrilled opening in a bone, the system comprising:
(a) at least one helical coil in a contracted state;
(b) an expansion tool operably engaging the helical coil to position the helical coil at a target site within the bone and to subsequently radially expand the helical coil to an expanded state allowing for removal of the expansion tool; and
(c) a threaded fastener threadedly inserted into the helical coil in the expanded state.

37. The implantable device of claim 2, wherein the threads of the helical fastener interdigitate with the threads on the inner surface of the helical coil.
